(19) United States
(12) Patent Application Publication
(10) Pub. No.: US 2022/0370508 A1
(43) Pub. Date: Nov. 24, 2022

(54) HUMAN UMBILICAL CORD COMPOSITIONS AND METHODS FOR INTRA-ARTICULAR THERAPY

(71) Applicant: BioStem Tehnologies, Inc., Pompano Beach, FL (US)

(72) Inventors: Wendy W. Weston, Coral Springs, FL (US); Michelle R. House, West Palm Beach, FL (US); Jason Matuszewski, Boca Raton, FL (US)

(21) Appl. No.: 17/873,383

(22) Filed: Jul. 26, 2022

Related U.S. Application Data
(63) Continuation-in-part of application No. 17/794,718, filed on Jan. 1, 0001.
(60) Provisional application No. 63/141,119, filed on Jan. 25, 2021.

(51) Int. Cl. A61K 35/51; A61K 9/00; A61K 31/728; A61K 38/17; A61K 38/18; A61K 38/20

(52) U.S. Cl. CPC A61K 35/51; A61K 9/0019; A61K 31/728; A61K 38/179; A61K 38/1833; A61K 38/2006; A61K 38/1858; A61K 38/1825

(57) ABSTRACT

An aqueous, non-immunogenic, injectable composition derived from human umbilical cords and methods of making thereof are described. The non-immunogenic composition is used for articular therapy in a human subject in need thereof. The non-immunogenic composition may include an aqueous human umbilical cord filtrate prepared without the use of exogenous enzymes resulting in exogenous enzymatic degradation/digestion.

### TECHNICAL FIELD

The present invention relates generally to the field of umbilical cord derived compositions, and more particularly to non-immunogenic compositions for intra-articular injection and treatment to a subject in need thereof in which the compositions are derived from fresh human umbilical cords.

### BACKGROUND

Compositions derived from human umbilical cords have various uses in the medical field. For example, these advantageous uses may include harvesting stem cells therefrom for the potential treatment of various blood diseases, cancers, and immune system disorders. When preparing compositions derived from human umbilical cords, the umbilical cord tissue is often subjected to harsh mechanical and enzymatic processing conditions in which specific cells (e.g., stem cells) may be isolated from the umbilical cord tissue, expanded/cultured, and cryopreserved, thus drastically altering the initial, endogenous cellular and extracellular profile of the umbilical cord tissue. Furthermore and either before, during, and/or after processing these umbilical cord isolates, exogenous additives, including various growth factors; cytokines such as interferon alpha (INF-α), are included within these isolates, which further alter these isolates when compared to the initial umbilical cord tissue. These alterations may further decrease efficacy in a desired treatment due to loss of the original, endogenous cellular and/or extracellular profile of the initial umbilical cord tissue.

### SUMMARY

In view of the above, it is an object of the invention to provide compositions derived from human umbilical cord(s) that mimic, include, and/or retain an extracellular profile similar to the endogenous profile of a human umbilical cord (e.g., in vivo), especially when compared with various previously mentioned umbilical cord isolates. These compositions described herein are prepared with fresh human umbilical cord (harvested and processed within 48 to 72 hours of extraction from the human subject) and, unlike the prior art compositions, are advantageously not subjected to biochemical and/or enzymatic digestion, which results in the compositions including and/or retaining a significant proportion of the extracellular profile (of a human umbilical cord in vivo).

In certain aspects, disclosed is an aqueous non-immunogenic injectable composition for intra-articular therapy in a human subject in need thereof. In certain aspects, the aqueous non-immunogenic composition comprises an aqueous human umbilical cord filtrate obtained from human umbilical cord. In certain aspects, the aqueous non-immunogenic composition consists of an aqueous human umbilical cord filtrate obtained from human umbilical cord. In certain aspects, the aqueous non-immunogenic composition consists essentially of an aqueous human umbilical cord filtrate obtained from human umbilical cord.

In certain aspects, the aqueous human umbilical cord filtrate is a solution in which no settling, separation, and/or precipitation is observed after twelve months, twenty-four months, up to 60 months or more while being stored at −20° C. or −80° C. In certain aspects, the aqueous human umbilical cord filtrate is a solution in which no settling, separation, and/or precipitation is observed after ten days, twenty days, thirty days, up to sixty days or more while being stored between 4° C. and 8° C. In certain aspects and when preparing the aqueous human umbilical cord filtrate, no exogenous enzymes are introduced therein, which avoids exogenous enzymatic degradation digestion. In certain aspects, the aqueous human umbilical cord filtrate is obtained by filtering ground human umbilical cord. In certain aspects, the aqueous non-immunogenic composition has particulates less than 100 μm. In certain aspects, the human umbilical cord is double-filtered to obtain aqueous human umbilical cord filtrate. In certain aspects the human umbilical cord is triple-filtered to obtain human umbilical cord filtrate. In certain aspects, the aqueous non-immunogenic injectable composition has particulates less than 50 μm. In certain aspects, the aqueous non-immunogenic injectable composition has particulates less than 35 μm. In certain aspects, the aqueous non-immunogenic injectable composition has particulates less than 10 μm. In certain aspects, the aqueous non-immunogenic injectable composition is sterile. In certain aspects, the aqueous non-immunogenic injectable composition is acellular.

In certain aspects, the aqueous human umbilical cord filtrate comprises at least one of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ interleukin antagonists (interleukin-1 receptor antagonist (IL-1ra)) at a concentration of 8.13×10² pg/mL to 5.15×10⁴ pg/mL, platelet derived growth factor BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least two of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ pg/mL, interleukin antagonists (interleukin-1 receptor antagonist (IL-1ra)) at a concentration of 8.13×10² pg/mL to 5.15×10⁴ pg/mL, platelet derived growth factor-BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least three of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ pg/mL, interleukin antagonists (interleukin-1 receptor antagonist (IL-1ra)) at a concentration of 8.13×10² pg/mL to 5.15×10⁴ pg/mL, platelet derived growth factor-BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least four of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL, to 1.42×10⁴ pg/mL, interleukin antagonists (interleukin-1 receptor antagonist (IL-1ra)) at a concentration of 8.13×10² pg/mL to 5.15×10⁴ pg/mL platelet derived growth factor-BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least five of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ pg/mL, interleukin antagonists (interleukin-1 receptor antagonist (IL-1ra)) at a concentration of 8.13×10² pg/mL to 5.15×10⁴ pg/mL, platelet derived growth factor-BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least six of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ pg/m, interleukin antagonists (interleukin-1 receptor antagonist (IL-1ra)) at a concentration of 8.13×10² pg/mL to 5.15×10⁴ pg/mL, platelet derived growth factor BB (PDGF-BB) at a concentration of 2.0×10¹ pg/mL to 1.58×10³ basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least seven of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ pg/, interleukin antagonists (interleukin-1 receptor antagonist (IL-1ra)) at a concentration of 8.13×10² pg/mL to 5.15×10⁴ pg/mL, platelet derived growth factor-BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate comprises at least eight of: acellular Wharton's jelly, exosomes, endogenous growth factors, vascular endothelial growth factor receptor-1 (VEGFR1) at a concentration of 1.0×10² pg/mL to 2.5×10³ pg/mL, hepatocyte growth factor (HGF) at a concentration of 2.5×10² pg/mL to 1.42×10⁴ pg/mL, interleukin antagonists (interleukin-1 receptor antagonist (IL-1ra)) at a concentration of 8.13×10² pg/mL to 5.15×10⁴ pg/mL, platelet derived growth factor-BB (PDGF-BB) at a concentration of 2.0×10¹ pg/mL to 1.58×10³ pg/mL, basic fibroblast growth factor (bFGF) at a concentration of 4.73×10¹ pg/ml to 2.07×10³ pg/ml, endogenous hyaluronic acid (HA) at a concentration of 1.51×10⁷ pg/mL to 3.5×10⁸ pg/mL, or any combination thereof.

In certain aspects, the aqueous human umbilical cord filtrate further includes an isotonic solution. In certain aspects, the isotonic solution is phosphate buffered saline (1× PBS), lactated ringers (sodium chloride 6 g/L, sodium lactate 3.1 g/L, potassium chloride 0.3 g/L, and calcium chloride 0.2 g/L at pH 6.5), isotonic saline (0.9 wt % sodium chloride), plasmalyte® (sodium chloride 5.26 g/L, potassium chloride 0.37 g/L, magnesium chloride hexahydrate 0.30 g/L, sodium acetate trihydrate 3.68 g/L, sodium gluconate 5.02 g/L at pH 7.4), or Normosol® (sodium chloride 5.26 g/L, KCl 0.37 g/L, magnesium chloride 0.30 g/L, sodium acetate anhydrous 122 g/L, sodium gluconate 5.02 g/L at pH 7.4). In certain aspects, the aqueous human umbilical cord filtrate further includes amniotic fluid.

In certain aspects, the filtrates mentioned immediately above may be further combined with an isotonic solution (a diluent) that may further dilute growth factor concentrations to a desired range. In certain aspects and when an isotonic solution is included therein, the composition comprises at least one of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived growth factor-BB (PDGF-BB) at a concentration ranging from 2.0×10¹ pg/mL to 1.05×10³ pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition comprises at least two of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived growth factor-BB (PDGF-BB) at a concentration ranging from 2.0×10¹ pg/mL to 1.05×10³ pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition. comprises at least three of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived growth factor-BB (PDGF-BB) at a concentration ranging from 2.0×10¹ pg/mL to 1.05×10² pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition comprises at least four of acellular Wharton's jelly, exosomes, endogenous growth factors, vascular endothelial growth factor receptor (VEGFR1) at a concentration ranging from 1.23×10² pg/mL to 1.9×1.0³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10² pg/mL to 3.43×10³ pg/mL, platelet derived growth factor-BB (PDGF-BB) at a concentration ranging from 2.0×10¹ pg/mL to 1.05×10² pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition comprises at least five of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived growth factor-BB (PDGF-BB) at a concentration ranging from 2.0×10¹ pg/mL to 1.05×10³ pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition comprises at least six of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HOF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived growth factor-BB (PDGF-BB) at a concentration ranging from 2.0×10¹ pg/mL to 1.05×10³ pg/mL, basic fibroblast growth factor (bFGF) at a concentration of 7.95×10¹ pg/mL to 1 .83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition comprises at least seven of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived growth factor-BB (PDGF-BB) at a concentration ranging from 2.0×10¹ pg/mL to 1.05×10³ pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects and when an isotonic solution is included therein, the composition comprises at least eight of acellular Wharton's jelly, exosomes, endogenous growth factors, vascular endothelial growth factor receptor (VEGFR1) at a concentration ranging from 1.23×10² pg/mL to 1.9×10³ pg/mL, hepatocyte growth factor (HGF) at a concentration ranging from 3.47×10² pg/mL to 1.0×10³ pg/mL, interleukin antagonists (interleukin-1 receptor antagonist (IL-1ra)) at a concentration ranging from 1.35×10³ pg/mL to 3.43×10³ pg/mL, platelet derived, growth factor-BB (PDGF-BB) at a concentration ranging from 2.0×10¹ pg/mL to 1.05×10³ pg/mL, basic fibroblast growth factor (bFGF) at a concentration of 7.95×10¹ pg/mL to 1.83×10³ pg/mL, endogenous hyaluronan (HA) at a concentration ranging from 1.51×10⁷ pg/mL to 1.0×10⁸ pg/mL, or any combination thereof.

In certain aspects, the composition further includes an effective amount of exogenous hyaluronic acid to restore endogenous extracellular matrix function in the intra-articular space, restore endogenous collagen function in the intra-articular space, and/or treat and/or reduce symptoms of inflammatory disease in the subject. In certain aspects, the exogenous hyaluronic acid is present in the composition at a concentration of 0.5 weight % to 5.0 weight %. In other aspects, the composition has a concentration of about 0.75 weight % to about 4.0 weight % exogenous hyaluronic acid. In other aspects, the composition has a concentration of about 1.5 weight % to about 3.5 weight %. In other aspects, the composition has a concentration of about 2 weight % to about 3.0 weight %.

In certain aspects, the composition is configured for intra-articular therapy in a human subject in need thereof as a sterile, injectable composition. In this aspect the composition is preferably a viscous aqueous composition having a sufficient viscosity for injection into an intra-articular space with minimal pain and/or discomfort to the subject and sufficient thickness and consistency to improve and/or repair and/or provide additional support to the extracellular matrix, promote water absorption into the extracellular matrix, and provide collagen in the intra-articular space thereby promoting joint cushioning and lubrication, and providing shock absorption to a subject's intra-articular space.

In certain aspects, also disclosed is a method of injecting the aqueous, non-immunogenic, injectable composition for articular therapy to a human subject in need thereof. The method includes step (a): injecting an effective amount of the composition to an intra-articular space of an affected joint in the human subject in need thereof to improve and/or restore endogenous extracellular matrix function in the intra-articular space, improve and/or restore endogenous collagen function in the intra-articular space, and/or treat and/or reduce symptoms of inflammatory disease in the subject, or any combination thereof.

In certain aspects, the composition is sterile. In certain aspects, the composition further comprises an effective amount of exogenous hyaluronic acid to improve and/or restore endogenous extracellular matrix function in the intra-articular space, improve and/or restore endogenous collagen function in the intra-articular space, to treat and/or reduce symptoms of inflammatory disease in the human subject in need thereof. In certain aspects, the exogenous hyaluronic acid is present in the composition at a concentration of 0.5 weight % to 5.0 weight %. In other aspects, the composition has a concentration of about 0.75 weight % to about 4.0 weight % exogenous hyaluronic acid. In other aspects, the composition has a concentration of about 1.5 weight % to about 3.5 weight %. In other aspects, the composition has a concentration of about 2 weight % to about 3.0 weight %.

In certain aspects, the articular therapy is delivered via intra-articular injection. This method comprises sterilely injecting the composition into and/or adjacent to the intra-articular space of the affected joint in the human subject in need thereof.

In certain aspects, injecting an effective amount of the composition to an affected joint of the human subject in need thereof to restore endogenous extracellular matrix function in the intra-articular space, restore endogenous collagen function in the intra-articular space, and/or treat and/or reduce symptoms of inflammatory disease in the subject is repeated at predetermined time intervals. In certain aspects, step (a) is repeated daily. In certain aspects, step (a) is repeated weekly. In certain aspects, step (a) is repeated monthly. In certain aspects, step (a) is repeated bi-weekly. In certain aspects, step (a) is repeated semi-weekly. In certain aspects, step (a) is repeated bi-monthly. In certain aspects, step (a) is repeated semi-monthly.

In certain aspects, the effective amount of composition to restore endogenous extracellular matrix function in the intra-articular space, restore endogenous collagen function in the intra-articular space, and/or treat and'or reduce symptoms of inflammatory disease in the. human subject is 0.5 mL. In certain aspects, the effective amount of composition to restore endogenous extracellular matrix function in the intra-articular space, restore endogenous collagen function in the intra-articular space, and/or treat and/or reduce symptoms of inflammatory disease in the human subject is 1 mL. In certain aspects, the effective amount of composition to restore endogenous extracellular matrix function in the intra-articular space, restore endogenous collagen function in the intra-articular space, and/or treat and/or reduce symptoms of inflammatory disease in the human subject is 2 mL. In certain aspects, the effective amount of composition to restore endogenous extracellular matrix function in the intra-articular space, restore endogenous collagen function in the intra-articular space, and/or treat and/or reduce symptoms of inflammatory disease in the human subject is 3 mL. In certain aspects, the effective amount of composition to restore endogenous extracellular matrix function in the intra-articular space, restore endogenous collagen function in the intra-articular space, and/or treat and/or reduce symptoms of inflammatory disease in the human subject is 4 mL. In certain aspects, the effective amount of composition to restore endogenous extracellular matrix function in the intra-articular space, restore endogenous collagen function in the intra-articular space, and/or treat and/or reduce symptoms of inflammatory disease in the human subject is 5 mL.

In certain aspects, the human subject in need thereof has osteoarthritis, rheumatoid arthritis, psoriatic arthritis, lupus, gout, or any combination thereof.

Embodiments of the invention can include one or more or any combination of the above features and configurations.

Additional features, aspects and advantages of the invention will be set forth in the detailed description which follows, and in part will be readily apparent to those skilled. In the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is a schematic depiction of the steps included for making the aqueous human umbilical cord filtrate of the injectable composition; and

FIG. 2 are graphs showing the concentration profiles of VEGFR1, HGF, interleukin antagonists (IL-1ra), bFGF, PDGF-BB and endogenous hyaluronan in the aqueous human umbilical cord filtrate.

### DETAILED DESCRIPTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings. Moreover, in this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

The compositions and methods described herein can comprise, consist of, or consist essentially of the essential elements and limitations described herein, as well as any additional or optional ingredients, components, or limitations described herein.

### Composition

Disclosed herein are compositions derived from human umbilical cord(s) that retain an extracellular profile similar to the endogenous profile of a human umbilical cord, for example, vivo, especially when compared with various previously mentioned umbilical cord isolates. These compositions are prepared with fresh human umbilical cord (harvested and processed within 48 to 72 hours of extraction from the human subject) and, unlike compositions in the prior art, are advantageously not subjected to biochemical and/or enzymatic digestion, which results in the compositions including and/or retaining a significant portion of the extracellular profile (when compared to the endogenous profile of a human umbilical cord in vivo). Moreover because of the ease and convenience of administering these compositions (e.g., point of use preparation and use within a dental office, medical office, or emergency room) and because of the non-immunogenic characteristics of these compositions, these compositions may be used for numerous different medical purposes and medical procedures, which include, but are not limited to, intra-articular therapy to a human subject in need thereof to improve and/or restore endogenous extracellular matrix function in the intra-articular space, improve and/or restore endogenous collagen function in the intra-articular space, to treat and/or reduce symptoms of inflammatory disease in the subject, or any combination thereof.

Disclosed herein are compositions (e.g., compositions) that include an aqueous human umbilical cord filtrate which may be configured for intra-articular therapy. In certain aspects and when preparing the composition no exogenous enzymes are introduced therein, which avoids exogenous enzymatic degradation/digestion and further ensures that these compositions have an improved endogenous extracellular profile (similar to human umbilical cord in vivo) especially when compared to conventional compositions utilizing umbilical cord tissues and/or cells derived therefrom.

Hyaluronic acid is the main component of the extracellular matrix and other human connective tissue and plays a number of structural roles in vivo. Endogenous hyaluronan and sulfated glycosaminoglycans (sGAGs) found within the composition described herein, increases the tensile strength of the extracellular matrix within the articular space. Additionally, hyaluronic acid may trigger intracellular events that lead to an increase in cell migration and proliferation. Injections of hyaluronic acid alone have been shown to restore the viscoelasticity in the joint. An additional property of hyaluronic acid is its ability to absorb water, or hygroscopicity. This property is desirable for articular therapy as it would draw water to the joint and provide further cushioning, lubrication, and shock absorption.

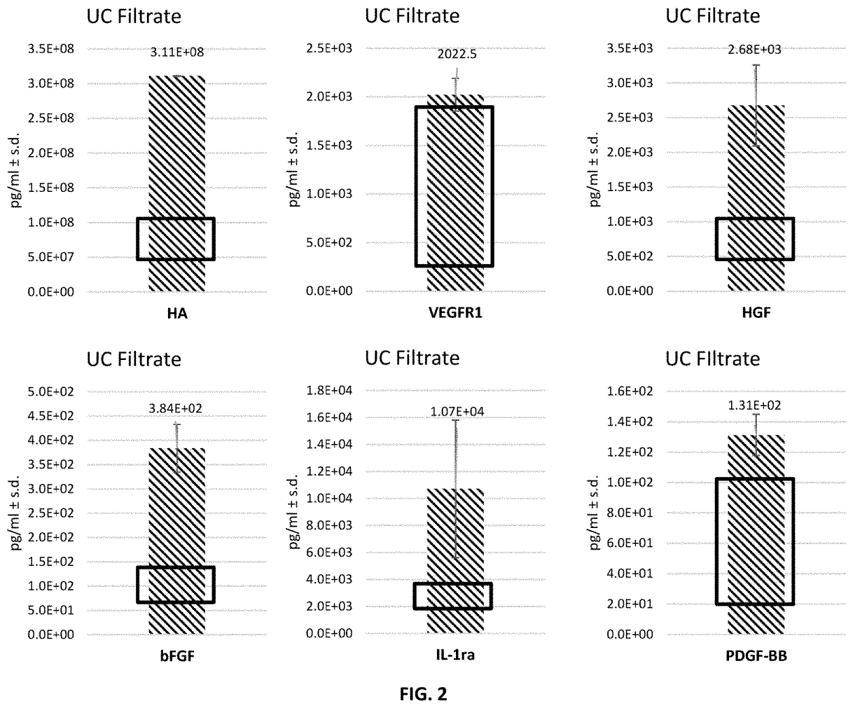

The aqueous human umbilical cord filtrate, of the component, is prepared, preferably from human umbilical cord via one or more separation steps (e.g., filtration steps). The human umbilical cord filtrate preferably includes acellular Wharton's jelly, exosomes, endogenous growth factors, vascular endothelial growth factor receptor 1 (VEGFR1), hepatocyte growth factor (HGF), interleukin antagonists (IL-1ra), platelet derived growth factor-BB (PDGF-BB), basic fibroblast growth factor (bFGF), endogenous hyaluronan (HA) or a combination thereof therein, which advantageously promotes joint lubrication and healing within a subject when the disclosed compositions are used for their desired purpose. As shown in FIG. 2, within the filtrate the concentrations of VEGFR1 ranges from 1.0×1.0² pg/mL to 2.5×10³ pg/mL, HGF ranges from 2.5×10² pg/mL to 1.42×10⁴ pg/mL, IL-1ra ranges from 8.13×10² pg/mL to 5.15×10⁴ pg/mL, PDGF-BB ranges from 2.0×10¹ pg/mL to 1.58×10³ pg/mL, bFGF ranges from 4.73×10¹ pg/ml to 2.07×10³ pg/ml, and HA ranges from 1.51×10⁷ pg/mL to 3.5×10⁸ pg/mL, and any combination thereof. In certain aspects, any endpoint falling within the above-mentioned ranges can serve as endpoints for any additional ranges falling in between.

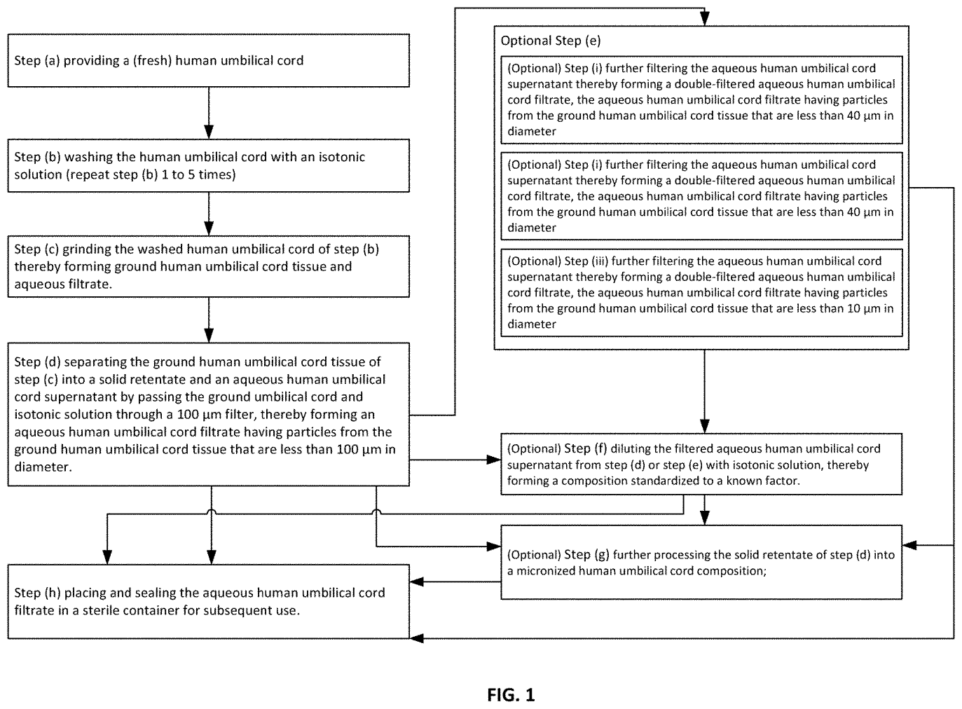

In certain aspects, the aqueous human umbilical cord filtrate may include particles that remain from a human umbilical cord tissue therein that are less than 100 μm in diameter, preferably less than 50 μm in diameter, more preferably less than 35 μm in diameter, even more preferably less than 10 μm in diameter. Moreover, aqueous human umbilical cord filtrate is a solution in which no settling, separation, and/or precipitation is observed after one month, two months, three months, four months, five months, six months, or more while being stored. In certain aspects and due to the preparation steps of these compositions as disclosed immediately below as well as in FIG. 1, the aqueous human umbilical cord filtrate further includes an isotonic solution such as phosphate buffered saline (or one of lactated ringers (sodium chloride 6 g/L, sodium lactate 3.1 g/L, potassium chloride 0.3 g/L, and CaCl 0.2 g/L at pH 6.5), isotonic saline (0.9 wt % sodium chloride), plasmalyte® (sodium chloride 5.26 g/L, potassium chloride 0.37 g/L, magnesium chloride hexahydrate 0.30 g/L, sodium acetate trihydrate 3.68 g/L, sodium gluconate 5.02 g/L at pH 7.4), Normosol® (sodium chloride 5.26 g/L, potassium chloride 0.37 g/L, magnesium chloride 0.30 g/L, sodium acetate anhydrous 2.22 g/L, sodium gluconate 5.02 g/L at pH 7.4)), which merely aids in the preparation of each component of the compositions disclosed herein and further has no and/or minimal degradative effects on, for example, acellular Wharton's jelly, exosomes, endogenous growth factors, VEGFR1, HGF, interleukin antagonists (e.g. IL-1ra), bFGF, PDGF-BB, endogenous hyaluronan or a combination thereof within the aqueous human umbilical cord filtrate. In certain aspects, it is envisioned that amniotic fluid may be used in addition to the aqueous human umbilical cord filtrate disclosed herein. Amniotic fluid may be used as a diluent in lieu of the isotonic solution. Amniotic fluid has a high concentration of human growth factor (HGF), which may be desired when using the disclosed composition. When any of the above isotonic solution(s) are added to the above disclosed filtrates, the isotonic solution(s) may act a diluent further diluting growth factor concentrations therein to a desired range. When an isotonic solution is added to the above disclosed filtrates, the concentrations of VEGFR1 ranges from 1.23×10² pg/mL to 1.9×10³ pg/mL, HGF ranges from 3.47×10² pg/mL to 1.0×10³ pg/mL, IL-1ra ranges from 1.35×10³ pg/ml to 3.43×10³ pg/mL, and PDGF-BB ranges from 2.00×10¹ pg/mL to 1.05×10² pg/mL, bFGF ranges from 7.95×10¹ pg/mL to 1.38×10² pg/mL, HA ranges from 1.51×10⁷ pg/mL to 1.0×10⁸ pg/mL, and any combination thereof. In certain aspects, any endpoint falling within the above-mentioned ranges can serve as endpoints for any additional ranges falling in between.

In certain aspects, exogenous hyaluronic acid may be added to the aqueous human umbilical cord filtrate. In certain aspects, the exogenous hyaluronic acid is present in the composition at a concentration of 0.5 weight % to 5.0 weight %. In other aspects, the composition has a concentration of about 0.75 weight % to about 4.0 weight % exogenous hyaluronic acid. In other aspects, the composition has a concentration of about 1.5 weight % to about 3.5 weight %. In other aspects, the composition has a concentration of about 2.0 weight % to about 3.0 weight %.

Hyaluronic acid is also referred to as hyaluronan or hyaluronate; these terms are used interchangeably throughout this specification. Hyaluronic acid is a glycosaminoglycan consisting of repeating units of D-glucoronic acid and N-acetyl-D-glucosamine. The hyaluronic acid used herein may be in salt form or non-salt form. Salt forms of hyaluronic acid include sodium hyaluronate, potassium hyaluronate, calcium hyaluronate, and magnesium hyaluronate. In some aspects, the hyaluronic acid used herein may be obtained from biofermentation in bacteria, including but not limited to: Enterococcus faecalis, Streptococcus zooepidernicus, Escherichia coli, Agrobacterium sp., Lactococcus lactis, and Bacillus subtilis. In other aspects, recombinant hyaluronic acid production is the source of the exogenous hyaluronic acid used herein. One example of recombinant hyaluronic acid production includes the expression hyaluronic acid synthase and UDP-glucose dehydrogenase in a host bacteria to produce large quantities of hyaluronic acid in a fed-batch culture process. In other aspects, the hyaluronic acid used herein may be obtained via extraction from animal tissues, including but not limited to: rooster combs, bovine synovial fluid, and vitreous humor of cattle. In some aspects, hyaluronic acid may be purchased from commercial sources, including but not limited to: Kewpie, Awa Biopharm, Dongchen Group, Fufeng Group, Focus Chem, and Bloomage Biotech.

The hyaluronic acid used herein may be in a variety of molecular weights. The term “molecular weight” may refer to both the weight-average molecular weight and the number-average molecular weight. In some aspects, the hyaluronic acid used herein may have a molecular weight of about 0.25 MDa to about 8.0 MDa.

In some aspects, the exogenous hyaluronic acid within the aqueous human umbilical cord filtrate is cross-linked. Hyaluronic acid may be cross-linked using a variety of cross-linking agents including, but not limited to, 1,4-butanediol diglycidyl ether (BDDE), poly (ethylene glycol) diglycidyl ether (PEGDE), pentaerythritol tetraglycidyl ether (PETGE), divinyl sulfone, 1,2-bis(2,3-epoxypropoxy)ethylene (EGDGE), 1,2,7,8-diepoxyoctane (DEO), (phenylenebis-(ethyl)-carbodiimde, 1,6-hexamethylenebis (ethylcarbodiimide), adipic dihydrazide (ADH), bis(sulfosuccinimdyl)suberate (BS), hexamethylenediiamine (HMDA), and 1-(2,3-epoxypropyl)-2,3-epoxycyclohexane. The degree of crosslinking, as used herein, is defined as the percent of free hyaluronic acid (non-cross-linked hyaluronic acid). In some aspects, the exogenous hyaluronic acid is heavily cross-linked, with a low percentage of tree hyaluronic acid, such as 5%-25%. In some aspects, the exogenous hyaluronic acid may be mildly cross-linked, about 26%-74% free hyaluronic acid. In some aspects, the exogenous hyaluronic acid may be lightly cross-linked, with a high percentage of free hyaluronic acid-75%-95% five hyaluronic acid. In some aspects, the hyaluronic acid is non cross-linked or 100% free hyaluronic acid. The degree of cross-linking within the hyaluronic acid increases the half-life of hyaluronic acid within the body, and thus longer therapeutic effects, such as joint cushioning and shock absorption, may be observed.

As further alluded to above, the compositions may be used as allografts within humans for numerous different purposes and procedures, which include, but are not limited to, intra-articular therapy. In this aspect, it is important to maintain sterility of the aqueous human umbilical cord filtrate. It should be noted that the aqueous human umbilical cord filtrate is non-immunogenic, and thus, should induce very little immune response within a subject when used for its desired purpose. However, sterility should be maintained such that contaminants (e.g., viral contaminants, bacterial contaminants, chemical contaminants, etc.) are not introduced into the composition that may induce an immune response and/or cause infection when the composition is placed in or on a subject.

In certain aspects, the compositions are configured for intra-articular therapy. In this aspect, the resulting composition is preferably a fluid having a sufficient consistency to lubricate, cushion, and/or provide shock absorption to the intra-articular space of a subject by replenishing structural proteins in the extracellular matrix, providing various growth factors and other nutrients from the composition, and in some aspects providing exogenous hygroscopic hyaluronic acid. For example, this composition may be injected into a subject's knee, ankle, hip, shoulder, or any other joint. It is also, envisioned that this composition may be injected into other joints, or intra-articular spaces, for substantially similar purposes. In another aspect, the composition is configured to be injected into a subject's heel or foot for the treatment of plantar fasciitis.

### Method of Making the Composition

FIG. 1 provides a schematic depiction of the steps included for making the aqueous human umbilical cord filtrate of the composition described herein, and as further shown in FIG. 1, none of steps include introduction of exogenous enzymes resulting in exogenous enzymatic degradation: digestion. The method of making the aqueous human umbilical cord filtrate configured for articular therapy, the method including steps (a)-(h) discussed immediately below. Before step (a), the umbilical cord and/or umbilical cord donor is screened for communicable diseases to ensure that the umbilical cord/umbilical cord tissue is healthy/disease free and to further minimize risk during preparation and subsequent end use of the compositions. The umbilical cord is maintained at temperature ranging from 4° C. to 8° C. before beginning the processing of the cord in steps (a)-(h).

As shown in FIG. 1, step (a) includes providing a human umbilical cord preferably within 24 to 96 hours post-extraction from a human subject, more preferably from 24 to 72 hours post-extraction from a human subject to ensure freshness of the human umbilical cord (i.e., tissue and cells comprising the tissue) and to minimize degradation resulting from necrosis, necroptosis and/or apoptosis. In this step and i.n order for appropriate grinding/mincing to occur (in subsequent step (c)), it is preferred that <80 grams is subject to the process at any one time.

After completing step (a), step (b) occurs. Step (b) includes placing <80 grams of umbilical cord into a container having a predetermined volume (e.g., 300 to 1000 mL, preferably 500 mL) of isotonic solution in which the isotonic solution is preferably phosphate buffered saline (PBS) (i.e., 1× PBS)(or alternatively one of lactated ringers (sodium chloride 6 g/L, sodium lactate 3.1 g/L, potassium chloride 0.3 g/L, and CaCl 0.2 g/L at pH 6.5), isotonic saline (0.9 wt % sodium chloride), plasmalyte® (sodium chloride 5.26 g/L, KCl 0.37 g/L, magnesium chloride hexahydrate 0.30 g/L, sodium acetate trihydrate 3.68 g/L, sodium gluconate 5.02 g/L at pH 7.4), Normosol® (sodium chloride 5.26 g/L, KCl 0.37 g/L, magnesium chloride 0.30 g/L, sodium acetate anhydrous 2.22 g/L, sodium gluconate 5.02 g/L at pH 7.4)). Placing the container onto a stir plate and placing a stir bar within the container (containing the PBS and umbilical cord) therein and stirring (medium to high speed) the umbilical cord within the isotonic solution for 5 to 15 minutes to wash the umbilical cord portions. Next, washing step (b) is repeated one to five times by decanting the “used” isotonic solution and pouring new isotonic solution into the container at a predetermined volume (e.g., 300 mL to 1000 mL, preferably 500 mL) to again wash the umbilical cord. Either before step (a), during step (a), after step (b), or during step (b) further determining whether any blood clots and/or blood pool(s)/pooling are present in the human umbilical cord and/or umbilical cord portions, and if so, removing these blood clots via suction or other mechanical removal means (e.g., scalpel, gauze and forceps) to further ensure that the presence of any immunogenic components (e.g., hemoglobin and/or home associated components from the umbilical cord donor) are minimized in the end resulting composition. During these washing steps, it is imperative to maintain an aseptic and/or sterile work environment to prevent and/or reduce introduction of any contaminants while making the composition.

Upon concluding step (b), step (c) is performed in which the washed umbilical cord is transferred to a grinding and/or mincing apparatus such as those disclosed in U.S. Pat. No. D716,601 “Tissue Mincing Tool” and/or U.S. Pat. No. 8,967,512 “Systems And Methods For Processing Cells”, which are incorporated by reference herein in their entirety, and a predetermined volume (e.g., 75 mL to 1.25 mL, preferably 100 mL) of the isotonic solution) is added to the apparatus. The washed umbilical cord is subsequently subjected to grinding and/or mincing by the grinding/mincing tool with the head of the grinding/mincing tool rotating at a range of 40 to 200 revolutions per minute (RPM) until the umbilical cord has been fully ground (or as close to fully ground as possible) thereby forming ground human umbilical cord tissue. During this grinding/mincing step, it is imperative to maintain a sterile work environment to prevent and/or reduce introduction of any contaminants while making the composition. In certain aspects, the grinding/mincing tool may be directly connected to an apparatus (i.e., a closed system environment as disclosed, for example, in U.S. Pat. No. 8,967,512) to further conduct steps (d) and/or (e) discussed below and to further maintain sterility and/or minimize the introduction of any contaminants while making the composition. Alternatively, steps (d) and/or (e) may be conducted in an open system laboratory environment.

Upon concluding step (c), step (d) is performed in which the ground/minced human umbilical cord tissue of step (c) is separated into a solid retentate and an aqueous human umbilical cord supernatant. This initial separation step may occur via a filtration process (either positive or negative pressure). For example, the minced/ground human umbilical cord tissue (of step (c) and included within a predetermined volume (e.g., 75 mL to 125 mL, preferably 100 mL) of the isotonic solution) may be placed directly on a filter having a desired porosity (e.g., 200 μm or 150 μm or 100 μm such as either a qualitative grade or quantitative grade mesh or net filter) and then. force (either positive or negative pressure) may or may not be applied such that a solid retentate (solids having a size above 200 μm or 150 μm or 100 μm) remain on the filter while an aqueous human umbilical cord supernatant (having any solids therein that are less than (200 μm or 150 μm or 100 μm) are passed through the filter. The filtration step generally takes 15 seconds to 2 minutes. Again, it is imperative to maintain a sterile and/or aseptic work environment to prevent and/or reduce introduction of any contaminants throughout step (d).

Upon concluding step (d), optional step (c) may be performed on the aqueous human umbilical cord supernatant. Step (e) preferably includes a plurality of filtration steps including: (i) filtering the aqueous human umbilical cord supernatant through a first filter having a porosity ranging from 30 μm to 40 μm thereby forming a second human umbilical cord supernatant; (ii) filtering the second human umbilical cord supernatant through a second filter having a porosity ranging from 10 μm to 25 μm thereby forming a third human umbilical cord supernatant; and (iii) filtering the third human umbilical cord supernatant through a third filter having a porosity ranging from 4 μm to 10 μm thereby forming the aqueous human umbilical cord filtrate. In certain aspects, the force applied is a negative pressure (vacuum) and preferred because such negative pressure is less likely to damage the filter and lead to subsequent quality control issues with the resulting compositions disclosed herein. The aqueous human umbilical cord filtrate preferably includes acellular Wharton's jelly, exosomes, endogenous growth factors, VEGFR1, HGF, interleukin antagonists IL-1ra), bFGF, PDGF-BB, endogenous hyaluronan, or any combination thereof. Each filtration step generally takes 15 seconds to 2 minutes at 1-5 psi vacuum to complete. Moreover, the resulting aqueous human umbilical cord filtrate from the above mentioned filtration steps is a solution in which no settling, separation, and/or precipitation is observed after one month, two months, three months, four months, five months, six months, twelve months, twenty-four months, sixty months, or)re while being stored. Upon filtering the human umbilical cord supernatant through a filter having a porosity of 10 μm or less, the solution is free from cells, resulting in an acellular supernatant. Therefore, in some aspects, the composition described herein is acellular.

Upon concluding step (d) and/or (e), optional step (f) may be performed where the human umbilical cord filtrate is diluted with an isotonic solution or amniotic fluid, thereby forming a composition standardized to a known factor (e.g. original umbilical cord weight, average growth factor content, etc.)

Upon concluding step (d) and/or (e), and either concurrently with (and/or either before or during step (e) and/or (f)), optional step (g) is performed that the solid retentate of step (d) is further processed into a micronized human umbilical cord composition by subjecting the solid retentate to a dehydration (lyophilization), freeze drying, and/or (cryomilling), process configured to yield particles (polydisperse particles) having sizes ranging from greater than 1 μm to 300 μm, preferably greater than 1 μm to 100 μm, and more preferably greater than 1 μm to 50 μm and more preferably greater than to than 1 μm to 35 μm. In certain aspects, step (e) is a cryomilling process (as described, for example, US 20160287749, US 20170203004, and U.S. Pat. No. 10,105,398, which are each incorporated by reference in their entirety herein) in which the solid retentate of step (d) is dehydrated and placed into a liquid nitrogen cooled cryomill chamber and subjected to grinding therein, thereby forming the micronized human umbilical cord composition having particle sizes ranging from greater than 1 μm to less than 300 μm, preferably greater than 1 μm to 100 μm, more preferably greater than 1 μm to 50 μm, and even more preferably from greater than 1 μm to 35 μm. The micronized human umbilical cord composition comprises collagen, fibronectin, endogenous hyaluronan, elastins, or any combination thereof. The micronized human umbilical cord may be saved for another application such as combining with the aqueous human umbilical cord filtrate of step (d), (e) or (f) to prepare a two-part composition for other envisioned therapeutic applications.

In certain aspects, after step (d), (e) and/or (f) is performed, exogenous hyaluronic acid is added to the aqueous human umbilical cord filtrate prior to step (h) below. In some aspects, heavily cross-linked hyaluronic acid is added to the aqueous human umbilical cord filtrate. In some aspects, mildly cross-linked hyaluronic acid is added to the aqueous human umbilical cord filtrate. In other aspects, lightly cross-linked hyaluronic acid is added to the aqueous human umbilical cord filtrate. In some aspects, non-cross-linked hyaluronic acid is added to the aqueous human umbilical cord filtrate. In certain aspects, exogenous hyaluronic acid is present in the composition at a concentration of 0.5 weight % to 5.0 weight %. In other aspects, the composition has a concentration of about 0.75 weight % to about 4.0 weight % exogenous hyaluronic acid. In other aspects, the composition has a concentration of about 1.5 weight % to about 3.5 weight %. In other aspects, the composition has a concentration of about 2 weight % to about 3.0 weight %.

In some aspects, the exogenous hyaluronic acid is added to the aqueous human umbilical cord filtrate as a solid. In other aspects, the exogenous hyaluronic acid is added to the aqueous human umbilical cord filtrate as an aqueous solution.

In certain aspects, the aqueous human umbilical cord filtrate is sterile, and the aqueous human umbilical cord filtrate is non-immunogenic.

After concluding step (d), (e) and/or (f), step (h) may be performed by placing and sealing the aqueous human umbilical cord filtrate (of step (d), (e) or (f) in a sterile container for subsequent use, wherein the aqueous human umbilical cord filtrate is sterile.

### Methods of Use

Without wishing to be bound by theory, it is envisioned that the compositions disclosed herein may be particularly useful for intra-articular therapy, and would advantageously produce very little immunogenic response due to the composition's non-immunogenic characteristics/properties.

Intra-articular therapy may be used in patients having soft tissue and connective tissue damage or degeneration and/or with various chronic illnesses, including but not limited to arthropathies such as osteoarthritis, rheumatoid arthritis, psoriatic arthritis, lupus, gout, and other arthritic conditions. Frequently, intra-articular therapy includes the injection of corticosteroids, analgesics, NSAIDs, and/or hyaluronic acid. Intra-articular therapy may be delivered via injection. into essentially any intra-articular space in the body. Most commonly, intra-articular therapy is delivered in the knee, hip, shoulder, and/or ankle however, other joint spaces may be candidates for intra-articular therapy as well, including but not limited to: finger and toe joints, wrist, elbow, jaw, spine, and neck.

Hyaluronic acid, found endogenously in the composition described herein, and in some aspects added exogenously to the composition, has also shown extreme efficacy as intra-articular therapy in various arthropathies. Hyaluronic acid is found naturally in the intra-articular synovial fluid and cartilage and serve as a shock absorber, protective coating, and lubricant on the articular cartilage surface. Many patients suffering from various arthropathies, such as osteoarthritis, have shown reduced concentration of hyaluronic acid in the intra-articular space. In addition to serving as a protective agent in the intra-articular space, hyaluronic acid has also shown to have an anti-inflammatory effect.

VEGFR1, HGF, interleukin antagonists (IL-1ra), bFGF and PDGF-BB, all present endogenously within the aqueous human umbilical cord filtrate, provide cell growth signaling and anti-inflammatory effects thereby providing various therapeutic effects for the relief of various joint ailments.

For example, it is envisioned that the compositions disclosed herein may be used for intra-articular therapy and more particularly to treat various arthropathies by restoring endogenous extracellular matrix function in the intra-articular space, restoring endogenous collagen function in the intra-articular space, and/or treating and/or reducing symptoms of inflammatory disease in the subject. In this particular use, the joint of the subject (knee, shoulder, ankle, wrist, elbow) are injected with the compositions disclosed herein.

In certain aspects, the composition may be injected into the desired intra-articular space at predetermined time intervals to achieve the desired results. In certain aspects, the compositions are injected daily. In certain aspects, the compositions are administered weekly. In certain aspects, the composition is injected monthly. In certain aspects, the composition is injected bi-weekly. In certain aspects, the composition is injected semi-weekly. In certain aspects, the composition is injected bi-monthly. In certain aspects, the composition is injected semi-monthly.

The compositions are delivered at an effective amount to restore endogenous extracellular matrix function in the intra-articular space, restore endogenous collagen function in the intra-articular space, and/or treat and/or reduce symptoms of inflammatory disease in the subject. The effective amount may be 0.5 mL to 5 mL, wherein any volumes falling therein may serve as endpoints for additional ranges.

In certain aspects, also disclosed are methods of treating an orthopedic and/or podiatric conditions/ailments. For example, in certain aspects, plantar fasciitis and/or heel ailments may be treated by injecting the composition disclosed herein directly into the subject's foot (subcutaneously in a portion between the ball and heel of the foot) and/or immediately adjacent to the portion of bone forming the subject's heel. This method comprises: sterilely injecting the mixed composition into and/or adjacent the area of the subject affected with orthopedic and/or podiatric conditions/ailments thereby treating the condition/ailment. In this aspect, the aqueous human umbilical cord filtrate is both sterile and non-immunogenic. For example, when treating one's plantar fasciitis with the above method and compositions, the compositions have sufficient thickness and viscosity to provide cushioning (subcutaneous cushioning) to treat and mitigate pain associated with plantar fasciitis. In particular, the Wharton's Jelly (mucopolysaccharides and proteoglycans) in the filtrate aid in the cushioning and protective purposes of the above-mentioned treatment(s).

In another example, it is further envisioned that the disclosed compositions may have more general applications in the medical field such as general wound packing (occurring in surgical procedures and/or acute trauma resulting in open external and/or internal wounds) and/or wound healing, in this aspect, the aqueous human umbilical cord filtrate may be used alone, or in combination with the micronized human umbilical cord. In some aspects, the micronized human umbilical cord may be combined with amniotic fluid in lieu of aqueous human umbilical cord filtrate. In these aspects, it is envisioned that one would initially assess the wound to generally determine the overall viscosity and thickness of the (mixed) two-part composition needed to, for example, pack and/or treat a subject's wound. Next, one sterilely mixes the composition to an effective viscosity to induce blood clotting; and then sterilely packs the subject's wound with the sterilely mixed composition to induce blood clotting within the sterilely packed wound. In certain aspects, the micronized human umbilical cord composition and the aqueous human umbilical cord filtrate are both sterile and non-immunogenic and are mixed at a ratio of 2:1 to 1:2 micronized human umbilical cord composition and the aqueous human umbilical cord filtrate during this method. If very viscous mixed composition is desired, a higher proportion of the micronized human umbilical cord composition is mixed with a lower proportion the aqueous human umbilical cord filtrate, and conversely, if a less viscous mixed composition is desired, a higher proportion of the aqueous human umbilical cord filtrate is mixed with a lower proportion of the micronized human umbilical cord composition. The above-mentioned packing may be repeated as necessary.

In certain aspects, each individual component of the two-part compositions disclosed herein may be used individually (alone) for specified purposes. For example, when using the disclosed filtrate individually, the purpose of using all filtrate (only filtrate) would be to provide the growth factors and exosomes within the filtrate as well as soluble scaffolding and stromal components. For example, if one were to use the filtrate to provide cushioning substance o a degenerative heel pad or intra-articular space. As another example and when using the disclosed micronized compositions individually (alone), the purpose of using all particulate would be to pack a wet wound bed or dental socket when the area is too wet to add additional filtrate, or another filtrate is desired, such as platelet rich plasma (PRP).

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. An aqueous, non-immunogenic, injectable composition for articular therapy in a human subject in need thereof, the composition comprising:
an aqueous human umbilical cord filtrate free of any exogenous enzymes and having particulates of less than 100 μm in the aqueous non-immunogenic composition.

2. The aqueous, non-immunogenic, injectable composition for articular therapy in a human subject in need thereof according to claim 1, wherein the aqueous human umbilical cord filtrate comprises at least four of:
(a) acellular Wharton's jelly,
(b) exosomes,
(c) endogenous growth factors,
(d) endogenous hyaluronan (HA) at a concentration ranging from 1.51×10⁷ pg/mL to 3.5×10⁸ pg/mL,
(e) vascular endothelial growth factor receptor (VEGFR1) at a concentration ranging from 1.0×10² pg/mL to 2.5×10³ pg/mL,
(f) hepatocyte growth factor (HGF) at a concentration ranging from 2.5×10² pg/mL to 1.42×10⁴ pg/mL,
(g) interleukin antagonists (interleukin-1 receptor antagonist (IL-1ra)) at a concentration ranging from 8.13×10² pg/mL to 5.15×10⁴ pg/mL,
(h) platelet derived growth factor-BB (PDGF-BB) at a concentration ranging from 2.0×10¹ pg/mL to 1.58×10³ pg/mL,
(i) basic fibroblast growth factor (bFGF) at a concentration of 4.73×10¹ pg/mL to 2.07×10³ pg/mL, or
(j) any combination thereof.

3. The aqueous, non-immunogenic, injectable composition for articular therapy in a human subject in need thereof according to claim 1, further comprising an isotonic solution.

4. The aqueous, non-immunogenic, injectable composition for articular therapy in a human subject in need thereof according to claim 3, wherein the isotonic solution is at least one of phosphate buffered saline; lactated ringers; a solution consisting essentially of sodium chloride, sodium acetate anhydrous, sodium gluconate, potassium chloride; and magnesium chloride, and a solution consisting essentially of sodium chloride, potassium chloride, magnesium chloride hexahydrate, sodium acetate trihydrate, and sodium gluconate.

5. The aqueous, non-immunogenic, injectable composition for articular therapy in a human subject in need thereof according to claim 4, wherein the aqueous non-immunogenic composition comprises at least four of:
(a) acellular Wharton's jelly,
(b) exosomes,
(c) endogenous growth factors,
(d) endogenous hyaluronan (HA) at a concentration ranging from 1.51×10⁷ pg/mL to 1.0×10⁸ pg/mL,
(e) vascular endothelial growth factor receptor (VEGFR1) at a concentration ranging from 1.23×10² pg/mL to 1.9×10³ pg/mL,
(f) hepatocyte growth factor (HGF) at a concentration ranging from 3.47×10² pg/mL to 1.0×1.0³ pg/mL,
(g) interleukin antagonists (interleukin-1 receptor antagonist (IL-1ra)) at a concentration ranging from 1.35×10³ pg/mL to 3.43×10³ pg/mL,
(h) platelet derived growth factor-BB (PDGF-BB) at a concentration ranging from 2.0×10¹ pg/mL to 1.05×10³ pg/mL,
(i) basic fibroblast growth factor (bFGF) at a concentration of 7.95×10¹ pg/mL to 1.83×10³ pg/mL, or
(j) any combination thereof.

6. The aqueous, non-immunogenic, injectable composition for articular therapy in a human subject in need thereof according to claim 1, wherein the aqueous non-immunogenic composition is acellular.

7. The aqueous, non-immunogenic, injectable composition for articular therapy in a human subject in need thereof according to claim 1, wherein the aqueous non-immunogenic composition further comprises an effective amount of exogenous hyaluronic acid to restore endogenous extracellular matrix function in the intra-articular space, restore endogenous collagen function in the intra-articular space, and/or treat and/or reduce symptoms of inflammatory disease in the subject.

8. The aqueous, non-immunogenic, injectable composition for articular therapy in a human subject in need thereof according to claim 7, wherein the aqueous non-immunogenic composition comprises exogenous hyaluronic acid at a concentration of 0.5 wt % to about 5.0 wt % of the overall composition.

9. The aqueous, non-immunogenic, injectable composition for articular therapy in a human subject in need thereof according to claim 1, wherein the aqueous human umbilical cord filtrate is sterile.

10. The aqueous, non-immunogenic, injectable composition for articular therapy in a human subject in need thereof according to claim 1, further comprising amniotic fluid.

11. The aqueous, non-immunogenic, injectable composition for articular therapy in a human subject in need thereof according to claim 1, wherein the aqueous non-immunogenic composition comprises particulates of less than 50 μm.

12. The aqueous, non-immunogenic, injectable composition for articular therapy in a human subject in need thereof according to claim 1, wherein the aqueous non-immunogenic composition comprises particulates of less than 35 μm.

13. The aqueous, non-immunogenic, injectable composition for articular therapy in a human subject in need thereof according to claim 1, wherein the aqueous non-immunogenic composition comprises particulates of less than 10 μm.

14. The aqueous, non-immunogenic, injectable composition for articular therapy in a human subject in need thereof according to claim 13, wherein the aqueous non-immunogenic composition is acellular.

15. A method of injecting the composition of claim 1 for articular therapy to a human subject in need thereof, the method comprising
(a) injecting an effective amount of the composition to an intra-articular space of an affected joint in the human subject in need thereof to improve and/or restore endogenous extracellular matrix function in the intra-articular space, improve and/or restore endogenous collagen function in the intra-articular space, to treat and/or reduce symptoms of inflammatory disease in the subject, or any combination thereof.

16. The method of claim 15, wherein the composition is sterile.

17. The method of claim 15, wherein the composition further comprises an effective amount of exogenous hyaluronic acid to improve and/or restore endogenous extracellular matrix function in the intra-articular space, improve and/or restore endogenous collagen function in the intra-articular space, to treat and/or reduce symptoms of inflammatory disease in the subject, or any combination thereof.

18. The method of claim 15, wherein step (a) is repeated at predetermined time intervals.

19. The method of claim 18, wherein step (a) is repeated daily, weekly, bi-weekly, semi-weekly, monthly, bi-monthly, or semi-monthly.

20. The method of claim 15, wherein the composition comprises at least four of:
(a) acellular Wharton's jelly,
(b) exosomes,
(c) endogenous growth factors,
(d) endogenous hyaluronan (HA) at a concentration ranging from 1.51×10⁷ pg/mL to 3.5×10 pg/mL,
(e) vascular endothelial growth factor receptor (VEGFR1) at a concentration ranging from 1.0×10² pg/mL to 2.5×10³ pg/mL,
(f) hepatocyte growth factor (HGF) at a concentration ranging from 2.5×10² pg/mL to 1.42×10⁴ pg/mL,
(g) interleukin antagonists (interleukin-1 receptor antagonist (IL-1ra)) at a concentration ranging from 8.13×10² pg/mL to 5.15×10⁴ pg/mL,
(h) platelet derived growth factor-RB (PDGF-BB) at a concentration ranging from 2.0×10¹ pg/mL, to 1.58×10³ pg/mL,
(i) basic fibroblast growth factor (bFGF) at a concentration of 4.73×10¹ pg/mL to 2.07×10³ pg/mL, or
(j) any combination thereof.

21. The method of claim 15, wherein the composition further comprises an isotonic solution.

22. The method of claim 21, wherein the composition comprises at least four of:
(a) acellular Wharton's jelly,
(b) exosomes,
(c) endogenous growth factors,
(d) endogenous hyaluronan (HA) at a concentration ranging from 1.51×10⁷ pg/mL to 1.0×10⁸ pg/mL,
(e) vascular endothelial growth factor receptor (VEGFR1) at a concentration ranging from 1.23×10² pg/mL to 1.9×10³ pg/mL,
(f) hepatocyte growth factor (HGF) at a concentration ranging from 3.47×10² pg/mL to 1.0×10³ pg/mL,
(g) interleukin antagonists (interleukin-1 receptor antagonist (IL-1ra)) at a concentration ranging from 1.35×10³ pg/mL to 3.43×10³ pg/mL,
(h) platelet derived growth factor-BR (PDGF-BB) at a concentration ranging from 2.0×10¹ pg/mL to 1.05×10³ pg/mL,
(i) basic fibroblast growth factor (bFGF) at a concentration of 7.95×10¹ pg/mL to 1.83×10³ pg/mL, or
(j) any combination thereof.

23. The method of claim 15, wherein the effective amount of the composition is selected from the group consisting of 0.5 mL, 1 mL, 2 mL, 3 mL, 4 mL, and 5 mL.

24. The method of claim 15, wherein the human subject in need thereof has at least one osteoarthritis, rheumatoid arthritis, psoriatic arthritis, lupus, gout, plantar fasciitis, or any combination thereof.
